(19) United States
(12) Patent Application Publication
(10) Pub. No.: US 2012/0157499 A1
(43) Pub. Date: Jun. 21, 2012

(54) DELIVERY OF AN ACTIVE DRUG TO THE POSTERIOR PART OF THE EYE VIA SUBCONJUNCTIVAL OR PERIOCULAR DELIVERY OF A PRODRUG

(75) Inventors: Patrick M. Hughes, Aliso Viejo, CA (US); Orest Olejnik, Coto De Caza, CA (US)

(73) Assignee: Allergan, Inc.

(21) Appl. No.: 13/407,906

(22) Filed: Feb. 29, 2012

Related U.S. Application Data
(63) Continuation of application No. 10/617,468, filed on Jul. 10, 2003.

(51) Int. Cl. A61K 31/4436; A61P 37/08; A61P 35/00; A61P 27/02; A61K 31/23

(52) U.S. Cl. 514/337; 514/552

(57) ABSTRACT

The present invention relates to method of sustained-delivery of an active drug to a posterior part of an eye of a mammal to treat or prevent a disease or condition affecting said mammal, wherein said disease or condition can be treated or prevented by the action of said active drug upon said posterior part of the eye, comprising administering an effective amount of an ester prodrug of the active drug subconjunctivally or periocularly. Preferably, the active drug is more than about 10 times as active as the prodrug. Other aspects of this invention deal with the treatment of certain diseases by the periocular or subconjunctival delivery of an ester prodrug, and certain pharmaceutical products containing ester prodrugs for periocular or subconjunctival administration.

### CROSS REFERENCE TO RELATED APPLICATIONS

This application is a continuation of U.S. patent application Ser. No. 10/617,468, filed on Jul. 10, 2003, and herein incorporated by reference.

### BACKGROUND OF THE INVENTION

1. Field of the Invention

The present invention relates to methods of delivering a drug. More particularly, the present invention relates to methods of delivering an active drug to a posterior part of the eye of a mammal.

2. Description of Related Art

There are many diseases or conditions which it is believed could be effectively treated or prevented by direct delivery of an active drug to posterior parts of the eye. Some examples of such diseases or conditions are retinitis pigmentosa, proliferative vitreal retinopathy (PVR), age-related macular degeneration (ARMD), diabetic retinopathy, diabetic macular edema, retinal detachment, retinal tear, uveitus, or cytomegalovirus retinitis. A major problem in the ophthalmic art is the difficulty in achieving effective delivery to posterior parts of the eye such as the uveal tract, vitreous, retina, choroid, optic nerve, or retinal pigmented epithelium to treat these diseases. The blood-retinal barriers provide a significant constraint to drug delivery to the posterior parts of the eye via topical or systemic administration. Furthermore, systemic administration of a drug intended to act in the posterior part of the eye requires administration of significantly larger quantities of the drug than would be necessary through targeted delivery. The result is an undesirably high systemic concentration of the drug, which is particularly problematic for toxic drugs, or those with undesirable side effects.

Circumventing blood-retinal barriers by direct intraocular administration using intra-ocular injections or implants is the current practice and thought to be the most efficient mode of delivery. Unfortunately, invasive techniques such as intraocular injection or implantation may result in retinal detachment, physical damage to the lens, as well as exogenous endophthalmitis. Direct intraocular injection or implantation also results in high pulsed concentrations of drug at the lens and other intraocular tissues, which carries significant risk, especially for drugs that possess intraocular toxicity. Furthermore, many drugs that are useful in treating conditions that affect the posterior parts of the eye are known to cause cataracts. Highly lipophilic drugs have the additional disadvantage of favorable partitioning into the lipophilic lens epithelium, further exacerbating their cataractogenic properties.

Furthermore, many drugs used to treat illnesses or conditions affecting the posterior part of the eye have very short intraocular half-lives. This requires that the drug be delivered frequently, or that the drug be delivered by a controlled-release delivery system. Frequent injection of a drug into the eye is highly undesirable for obvious reasons, so controlled-release or sustained release delivery is generally used. For example, intrascleral injection of an active drug incorporated into a biodegradable or biocompatible polymer for the controlled-release or sustained release of drugs targeted to the back of the eye has been reported in the patent literature (U.S. Pat. No. 6,378,526 and U.S. Pat. No. 6,397,849). Often the polymers are used in the form of microparticles for the controlled-release of ophthalmic drugs. Generally, the microparticle consists of the drug entrapped in a polymer (see Joshi, “Microparticles for Ophthalmic Drug Delivery”, Journal of Ocular Pharmacology, Vol. 10, No. 1, 1994, pp. 29-45).

The drug is slowly released by mechanisms such as degradation or dissolution of the polymer, erosion, diffusion, ion-exchange, or a combination thereof. Einmal and coworkers (“A Novel Route of Ocular Drug Delivery: Suprachoroidal Injections Of A Sustained-Release System”, Proceed. Intl. Symp. Rel. Bioact. Mater., 28, (2001), pp. 293-294) have further shown that suprachoroidal injection of poly(orthoester) loaded with magnesium hydroxide and dexamethasone phosphate provided sustained delivery of the drug to the choroid and the retina.

The concept of prodrugs is well known in the art, and prodrugs have been used to improve the physical, chemical, and biological properties of drugs suffering from defects that affect their suitability for use in treating human or animal disease. A prodrug might be used, for example, to alter the hydrophobicity or lipophilicity of a drug to allow it to more readily penetrate a biological barrier, increase solubility, stabilize a drug so that it can reach its physiological target, reduce the occurrence of side effects, improve the shelf life of a drug, or aid in formulation. Generally speaking, prodrugs are derivatives of physiologically active drugs, which after administration undergo conversion to the active species. The conversion may be enzyme catalyzed, but it is also possible for the prodrug to be unstable to hydrolysis or some other reaction in a physiological environment. From among the voluminous scientific literature devoted to prodrugs in general, the foregoing examples are cited: Design of Prodrugs (Bundgaard H. ed.) 1985 Elsevier Science Publishers B. V. (Biomedical Division), Chapter 1; Design of Prodrugs: Bioreversible derivatives for various functional groups and chemical entities (Hans Bundgaard); Bundgaard et al. Int. J. of Pharmaceutics 22 (1984) 45-56 (Elsevier); Bundgaard et al. Int. J. of Pharmaceutics 29 (1986) 19-28 (Elsevier); Bundgaard et al. J. Med. Chem. 32 (1989) 2503-2507 Chem. Abstracts 93, 137935y (Bundgaard et al.); Chem. Abstracts 95, 138493f (Bundgaard et al.); Chem. Abstracts 95, 138592n (Bundgaard et al.); Chem. Abstracts 110, 57664p (Alminger et al.); Chem. Abstracts 115, 64029s (Buur et al.); Chem. Abstracts 115, 189582y (Hansen et al.); Chem. Abstracts 117, 14347q (Bundgaard et al.); Chem. Abstracts 117, 55790x (Jensen et al.); and Chem. Abstracts 123, 17593b (Thomsen et al.).

### SUMMARY OF THE INVENTION

The present invention relates to the use of a prodrug to increase the duration of action of an active drug in the eye. When prodrugs are used to increase the duration of action of an active drug, the necessity of administering a large amount of the prodrug relative to the therapeutically effective amount of the active drug is often a significant disadvantage. In other words, when a long duration of action is desired, a large amount of the active drug is “stored” as the prodrug, so a high concentration of prodrug will be present in the system. If the prodrug is more toxic or has more unpleasant side effects than the active drug, this is particularly problematic and becomes worse as the desired duration of action increases because a larger amount of prodrug is required. The present invention reduces this significant disadvantage associated with the use of a prodrug in the eye by administration of the prodrug in such a way as to reduce the amount of the prodrug required to be present in the eye to achieve sustained therapeutic concentrations of the active drug in the eye.

We have surprisingly discovered that an active drug can actually be delivered to the vitreous and other posterior parts of the eye by subconjunctival or periocular administration of an ester prodrug more efficiently than by direct intraocular administration of the ester prodrug. In other words, when a prodrug is administered subconjunctivally or periocularly, the ratio of the prodrug to active drug is significantly lower in the eye than it is when the prodrug is administered intraocularly or directly into the vitreous. As a result, sustained delivery of therapeutically-effective concentrations of the active drug to the posterior parts of the eye can be achieved with fewer side effects such as cataracts, and a lower risk of toxicity associated with the prodrug, by subconjunctival or periocular administration of the prodrug instead of direct intraocular or intravitreal administration of the prodrug. As such, this invention dramatically improves the pharmacotherapy of compounds with low therapeutic indices directed at the posterior ocular structures.

This invention also relates to the treatment of certain diseases by the periocular or subconjunctival delivery of an ester prodrug and certain pharmaceutical products containing ester prodrugs for periocular or subconjunctival administration.

### BRIEF DESCRIPTION OF THE DRAWING FIGURES

FIG. 1 shows tazarotene concentration (mean±standard deviation) in aqueous humor, vitreous humor, and retina (N=4) after a single subconjunctival injection of 1 mg tazarotene in a suspension. The mean represents the average concentration of tazarotene in the respective tissues measured in 4 different eyes at each time point.

FIG. 2 shows tazarotenic acid concentration (mean±standard deviation) in aqueous humor, vitreous humor, and retina (N=4) after a single subconjunctival injection of 1 mg tazarotene in a suspension. The mean represents the average concentration of tazarotenic acid in the respective tissues measured in 4 different eyes at each time point.

FIG. 3 shows tazarotene concentration (mean±standard deviation) in aqueous humor, vitreous humor, and retina (N=4) after a single subconjunctival injection of 1 mg tazarotene in a solution. The mean represents the average concentration of tazarotene in the respective tissues measured in 4 different eyes at each time point.

FIG. 4 shows tazarotenic acid concentration (mean±standard deviation) in aqueous humor, vitreous humor, and retina (N=4) after a single subconjunctival injection of 1 mg tazarotene in a solution. The mean represents the average concentration of tazarotenic acid in the respective tissues measured in 4 different eyes at each time point.

FIG. 5 shows tazarotene concentration (mean±standard deviation) in aqueous humor, vitreous humor, and retina (N=4) after a single subconjunctival injection of 0.5 mg tazarotene in poly(lactide-co-glycolide) (PGLA) microspheres. The mean represents the average concentration of tazarotene in the respective tissues measured in 4 different eyes at each time point.

FIG. 6 shows tazarotenic acid concentration (mean±SD) in aqueous humor, vitreous humor, and retina (N=4) after a single subconjunctival injection of 0.5 mg tazarotene in PGLA microspheres. The mean represents the average concentration of tazarotenic acid in the respective tissues measured in 4 different eyes at each time point.

FIG. 7 shows intravitreal concentrations of tazarotene and tazarotenic acid intravitreal administration of tazarotene.

FIG. 8 shows vitreous tazarotene/tazarotenic acid concentration ratios by mode of administration: 1. Subconjunctival suspension, 2. Subconjunctival oil, 3. Subconjunctival microsphere, 4. Intravitreal injection

FIGS. 9 and 10 are representations of the human eye which illustrate where the prodrug may be administered.

### DETAILED DESCRIPTION OF THE INVENTION

This invention relates to a method of sustained-delivery of an active drug to a posterior part of an eye of a mammal to treat or prevent a disease or condition affecting said mammal, wherein said condition can be treated or prevented by the action of said active drug upon said posterior part of the eye, comprising administering an effective amount of an ester prodrug of the active drug subconjunctivally or periocularly. Preferably, the active drug is more than about 10 times as active as the prodrug. It is also preferred that the active drug is not a platelet activating factor antagonist.

The phrase “posterior part of the eye” is defined as an area of the eye comprising one particular part of the posterior of the eye, a general region in the posterior part of the eye, or a combination of the two. Preferably the posterior part of the eye being acted upon by the active drug comprises the uveal tract, vitreous, retina, choroid, optic nerve, or retinal pigmented epithelium. The disease or condition related to this invention comprises any disease or condition that can be prevented or treated by the action of the active drug upon a posterior part of the eye. While not intending to limit the scope of this invention in any way, some examples diseases or conditions can be prevented or treated by the action of an active drug upon the posterior part of the eye include maculopathies/retinal degeneration such as non-exudative age related macular degeneration (ARMD), exudative age related macular degeneration (ARMD), choroidal neovascularization, diabetic retinopathy, acute macular neuroretinopathy, central serous chorioretinopathy, cystoid macular edema, and diabetic macular edema; uveitis/retinitis/choroiditis such as acute multifocal placoid pigment epitheliopathy, Behcet's disease, birdshot retinochoroidopathy, infectious (syphilis, lyme, tuberculosis, toxoplasmosis), intermediate uveitis (pars planitis), multifocal choroiditis, multiple evanescent white dot syndrome (mewds), ocular sarcoidosis, posterior scleritis, serpiginous choroiditis, subretinal fibrosis and uveitis syndrome, Vogt-Koyanagi- and Harada syndrome; vascular diseases/exudative diseases such as retinal arterial occlusive disease, central retinal vein occlusion, disseminated intravascular coagulopathy, branch retinal vein occlusion, hypertensive fundus changes, ocular ischemic syndrome, retinal arterial microaneurysms, Coat's disease, parafoveal telangiectasis, hemi-retinal vein occlusion, papillophlebitis, central retinal artery occlusion, branch retinal artery occlusion, carotid artery disease (CAD), frosted branch angiitis, sickle cell retinopathy and other hemoglobinopathies, angioid streaks, familial exudative vitreoretinopathy, and Eales disease; traumatic/surgical conditions such as sympathetic ophthalmia, uveitic retinal disease, retinal detachment, trauma, conditions caused by laser, conditions caused by photodynamic therapy, photocoagulation, hypoperfusion during surgery, radiation retinopathy, and bone marrow transplant retinopathy; proliferative disorders such as proliferative vitreal retinopathy and epiretinal membranes, and proliferative diabetic retinopathy; infectious disorders such as ocular histoplasmosis, ocular toxocariasis, presumed ocular histoplasmosis syndrome (POHS), endophthalmitis, toxoplasmosis, retinal diseases associated with HIV infection, choroidal disease associate with HIV infection, uveitic disease associate with HIV infection, viral retinitis, acute retinal necrosis, progressive outer retinal necrosis, fungal retinal diseases, ocular syphilis, ocular tuberculosis, diffuse unilateral subacute neuroretinitis, and myiasis; genetic disorders such as retinitis pigmentosa, systemic disorders with accosiated retinal dystrophies, congenital stationary night blindness, cone dystrophies, Stargardt's disease and fundus flavimaculatus, Best's disease, pattern dystrophy of the retinal pigmented epithelium, X-linked retinoschisis, Sorsby's fundus dystrophy, benign concentric maculopathy, Bietti's crystalline dystrophy, and pseudoxanthoma elasticum; retinal tears/holes such as retinal detachment, macular hole, and giant retinal tear; tumors such as retinal disease associated with tumors, congenital hypertrophy of the retinal pigmented epithelium, posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, and intraocular lymphoid tumors; and miscellaneous other diseases affecting the posterior part of the eye such as punctate inner choroidopathy, acute posterior multifocal placoid pigment epitheliopathy, myopic retinal degeneration, and acute retinal pigment epitheliitis. Preferably, the disease or condition is retinitis pigmentosa, proliferative vitreal retinopathy (PVR), age-related macular degeneration (ARMD), diabetic retinopathy, diabetic macular edema, retinal detachment, retinal tear, uveitus, or cytomegalovirus retinitis.

An ester prodrug is a prodrug having the meaning described previously, which is also an ester. The ester functional group is responsible for the activation-deactivation properties of the active drug. In other words, the prodrug yields the active drug as an alcohol or acid upon hydrolysis of the ester functional group.

While not intending to be bound by any theory, it is believed that higher esterase activity in the choroid and iris-ciliary body relative to the vitreous allows a higher ratio of active drug to prodrug to be delivered to the vitreous via subconjunctival or periocular injection than can be achieved by direct injection of the prodrug into the vitreous. It is also believed that the subconjunctival or periocular space can serve as a depot for an ester prodrug, thus allowing sustained delivery of the drug to the back of the eye while avoiding a high concentration of the prodrug in either the eye or the whole body. In other words, targeted delivery of the active drug is accomplished by indirect administration of the prodrug. Generally, without targeted delivery, administration of a prodrug systemically would require high systemic concentration of the prodrug so that a therapeutically effective amount of the active drug is present in the back of the eye. This scenario has great potential for unacceptable side effects. In this invention, the delivery of the active drug is targeted, but the prodrug is not administered to the site of action or to the sensitive surrounding areas. Rather the prodrug is administered to an area near enough to the site of action to have therapeutically effective targeted delivery, but far enough from the particularly sensitive parts of the eye that harmful side effects are reduced significantly. Thus this invention allows a therapeutic concentration of the active drug to be available to the posterior parts of the eye for a sustained period of time, while the concentration of the prodrug in the sensitive parts of the eye and the entire body of the mammal are significantly reduced.

The ester prodrug can be any ester which fits the criteria described above. Preferably, the prodrug is a carboxylic acid ester. While not intending to be limiting, it is known in the art that the cornea and iris-ciliary body are rich in esterases, so a carboxylic acid ester that can be used topically on the cornea to treat a disease where the drug acts in the interior of the eye is a prodrug of one of the hydrolysis products. In a preferred embodiment of this invention, the ester group of the prodrug which is hydrolyzed to form the active drug is not a lactone, or a cyclic carboxylic acid ester. In another preferred embodiment of this invention the prodrug is an ester of a phosphorous or sulfur-based acid.

In relation to this invention, the active drug is more than about ten times as active as the prodrug in an appropriate assay. An appropriate assay is one that is accepted by a person of ordinary skill in the art to be relevant to the disease or condition to be treated or prevented. Additionally, an appropriate assay should also distinguish between the prodrug and the active drug, meaning that the two compounds give significantly different results in the assay. While not intending to limit the scope of the invention in any way, suitable assays are receptor binding assays, activity assays, or other in vitro assays. In the case of binding or activity related to biological receptors, the assay could be relevant to a single receptor or receptor subtype or to more than one receptor or receptor subtype.

While not intending to be limiting, some relevant receptor targets are retinoid receptors, including RAR subtypes α, β, and γ, RXR subtypes α, β, and γ, VEGFR and other tyrosine kinase receptors, alpha adrenergic receptors, alpha 2 adrenergic receptors and subtypes 2A, 2B and 2C, beta adrenergic receptors, cholinergic receptors, muscarinic receptors, integrin receptors αᵥβ3 and αᵥβ5, and the steroid receptor subfamily of the nuclear receptors.

In cases where a relevant receptor assay is not known, or where it is known that there is no relevant receptor, a suitable functional assay is used. The functional assay used should be accepted in the art to be relevant to the condition or disease being treated or prevented. The functional assay should also be able to distinguish between the prodrug and the active drug, meaning that the two compounds give significantly different results in the assay. For example, while not intending to limit the scope of the invention, in the case of antibiotics, a suitable efficacy test can be used such as the disc diffusion method where the zone of inhibition indicates a ten fold less potency for the prodrug compared to the active drug. In the case of neurotoxins, the mouse potency assay can be used as a measure of potency. Similarly for any other disease or condition and active drug where a receptor-binding assay does not exist or is not relevant, a suitable functional assay is used. In the case that more than one assay is applicable to the disease, the prodrug need only be more than about ten times more active than the active drug in one of the assays.

The active drug of this invention could be any type of drug, useful in treating a disease or condition affecting the back of the eye, which could be formed by hydrolysis of an ester prodrug under biological conditions. Preferred active drugs are retinoids, prostaglandins, alpha-2-adrenergic agonists, beta adrenoreceptor antagonists, dopaminergic agonists, cholenergic agonists, tyrosine kinase inhibitors, antiinflammatories, corticosteroids, NMDA antagonists, anti-cancer drugs and antihistamines. In a preferred embodiment of this invention, the active drug is a retinoid. A retinoid is defined as a compound having retinoid-like activity. Compounds which have retinoid activity are well known in the art, and are described in numerous patents in the United States and other countries, as well as in numerous scientific publications. While not intending to limit the scope of this invention in any way, some examples of retinoids which are active drugs in this invention are 13-cis-retinoic acid, 13-cis-retinol, all-trans-retinoic acid, all-trans retinol. A particularly useful retinoid, which is the active drug in a more preferred embodiment of this invention, is 4,4-dimethyl-6-[2′-(5″-carboxy-2″-pyridyl)-ethynyl]-thiochroman, otherwise known as tazarotenic acid, which has the structure shown in Formula I below.

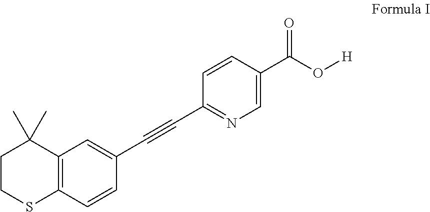

As mentioned previously, the active drug is a hydrolysis product of the prodrug. Since ester hydrolysis yields both an acid and an alcohol, the active drug could be either the acid or the alcohol hydrolysis product. The acid hydrolysis product could be a carboxylic acid, or another organic acid such as a sulfur or phosphorous based acid. Additionally, the acid component can breakdown into further components (e.g. acyloxyalkyl prodrugs). Since many acids are deprotonated under physiological conditions, the active drug may also be a salt of one of the organic acids formed from hydrolysis. The salt of the organic acid should be broadly interpreted to mean the dissociated anion formed by deprotonation, the ion pair, or any form that is not completely dissociated or tightly paired. Preferably, the active drug is a carboxylic acid, a carboxylic acid salt, or an alcohol.

In a preferred embodiment of this invention, the prodrug is an ester of the active drug, wherein the active drug is a carboxylic acid or salt thereof. More preferred prodrugs are those consisting of an ester formed from the active drug which is a carboxylic acid or salt thereof, and a C₁₋₆ alcohol or phenol. More preferred are prodrugs which are ethyl esters of an active drug which is a carboxylic acid or salt thereof. In the most preferred embodiment of this invention, the prodrug is ethyl 6-[(4,4-dimethylthiochroman-6-yl)ethynyl]nicotinate, otherwise known as tazarotene, which is the ethyl ester of the previously described tazarotenic acid.

In a preferred embodiment of this invention, the prodrug or active drug is cataractogenic. A cataractogenic active drug or prodrug causes or contributes to the medical condition affecting the eye known as cataracts.

In another embodiment of this invention, the prodrug is contained in a polymeric microparticle system designed to enhance the sustained-delivery of said active drug. While not intending to limit the scope of the invention in any way, microparticle systems designed to enhance the sustained-delivery of a drug are well known in the art, and there are a number of methods known in the art for preparing these drug-containing polymer microparticle systems. In a preferred embodiment of this invention, the polymeric microparticle system is a poly(lactide-co-glycolide) (PLGA) microsphere suspension.

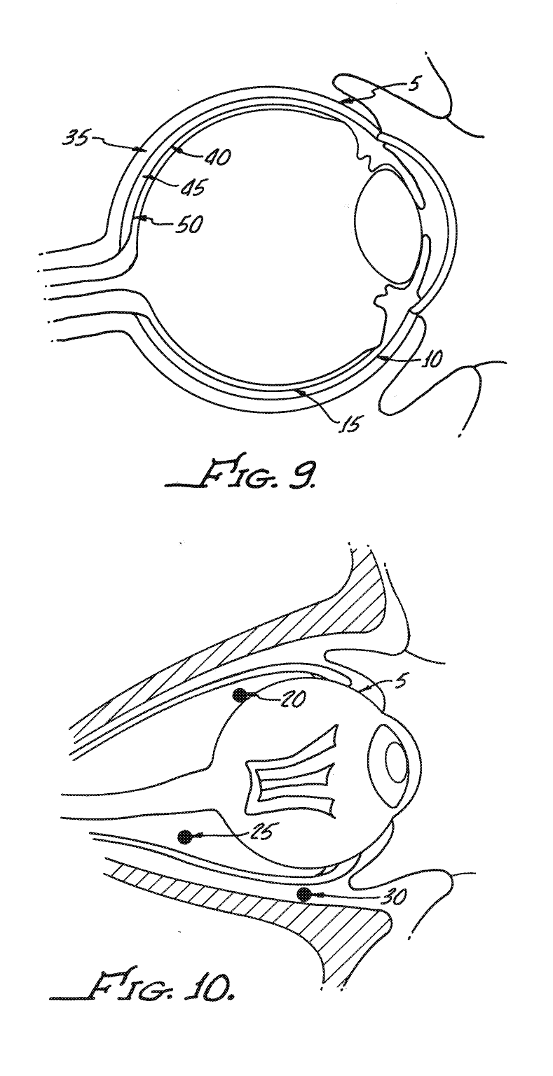

The prodrug is administered subconjunctivally or periocularly. Turning to FIG. 9, the retinal pigmented epithelium 40, choroid 45, and schlera 35 are indicated in the diagram. Administration of the prodrug can be subconjunctival 5, schlera 10, or supra-choroidal 15. Turning to FIG. 10, administration of the prodrug can also be sub-tenon 20, retrobulbar 25, or peribulbar 30.

Preferably, administration is subconjunctival 5. Administration could be carried out by injection, implant or an equivalent method. Preferably, administration is carried out via injection. Another embodiment of this invention relates to a method of treating or preventing a disease or condition, wherein treatment or prevention of said disease or condition is achieved by the action of an active drug on a posterior part of an eye of an affected mammal, comprising administering an effective amount of a carboxylic acid ester prodrug of the active drug subconjunctivally or periocularly via injection, wherein the prodrug is contained in a polymeric microparticle system designed to enhance the sustained-delivery of said active drug wherein the active drug is more than about 10 times as active as the prodrug.

Another embodiment of this invention relates to a pharmaceutical product comprising
i) a composition containing an effective concentration of an ester prodrug of an active drug, wherein the action of said active drug on a posterior part of an eye of a mammal is effective in treating or preventing a disease or condition affecting said posterior part of the eye, and wherein the active drug is more than about 10 times as active as the prodrug; and
ii) a suitable packaging material which comprises instructions that the product is to be used to treat said disease or condition by injecting said product subconjunctivally or periocularly, wherein said instructions do not indicate that the product is to be administered by intravitreal or intraocular injection or wherein said instructions indicate or suggest a preference for subconjunctival or periocular injection over intravitreal or intraocular injection.

The term “packaging material” comprises any container which holds the composition containing the carboxylic ester prodrug, as well as any auxiliary packaging around said container. While not intending to limit the scope of the invention in any way, the auxiliary packaging could comprise a box, shrink wrap, paper wrap, or the like. The auxiliary packaging also comprises any material prepared by or for the manufacturer of the pharmaceutical product, which is designed to aid the physician or the patient in the use of the product. This auxiliary packaging does not necessarily have to be physically sold or distributed with the product. The instructions referred to could be written, illustrated by figures, drawings, diagrams and the like, or a combination thereof, and could be contained on any part of the packaging material considered in its broadest sense. Additionally, the instructions could be verbally or visually contained on a recorded medium such as an audiotape or videotape, compact disk, or DVD.

A person skilled in the art will recognize that there are many ways in which the preferences or embodiments described above can be combined to form unique embodiments. Any combination of the preferences or embodiments mentioned herein which would be obvious to those of ordinary skill in the art are considered to be separate embodiments which fall within the scope of this invention.

The best mode of making and using the present invention are described in the following examples. These examples are given only to provide direction and guidance in how to make and use the invention, and are not intended to limit the scope of the invention in any way.

### Example A

The binding of tazarotene and tazarotenic acid to the retinoic acid receptor (RAR) family receptors (RAR_{α}, RAR_{β}, RAR_{γ}) was determined as follows.

All binding assays were performed in a similar fashion. All three receptor subtypes were derived from the expressed receptor type (RAR_{α}, RAR_{β}, and RAR_{γ}) expressed in Baculovirus. Stock solutions of the compounds were prepared as 10 mM ethanol solutions and serial dilutions carried out into 1:1 DMSO; ethanol. Assay buffers consisted of the following for all six receptor assays: 8% glycerol, 120 mM KCl, 8 mM Tris, 5 mM CHAPS 4 mM DTT and 0.24 mM PMSF, pH-7.4 @ room temperature.

All receptor binding assays were performed in the same manner. The final assay volume was 250 μl and contained from 10-40 μg of extract protein depending on receptor being assayed along with 5 nM of [³H] all-trans retinoic acid or 10 nM [³H] 9-cis retinoic acid and varying concentrations of competing ligand at concentrations that ranged from 0-10⁵M. The assays were formatted for a 96 well minitube system. Incubations were carried out at 4° C. until equilibrium was achieved. Non-specific binding was defined as that binding remaining in the presence of 1000 nM of the appropriate unlabeled retinoic acid isomer. At the end of the incubation period, 50. mu.l of 6.25% hydroxyapitite was added in the appropriate wash buffer. The wash buffer consisted of 100 mM KCl, 10 mM Tris and either 5 mM CHAPS (RAR_{α}, RAR_{β}, and RAR_{γ}) or 0.5% Triton X-100 (RAR_{α}, RAR_{β}, and RAR_{γ}). The mixture was vortexed and incubated for 10 minutes at 4° C., centrifuged and the supernatant removed. The hydroxyapitite was washed three more times with the appropriate wash buffer. The receptor-ligand complex was adsorbed by the hydroxyapitite. The amount of receptor-ligand complex was determined by liquid scintillation counting of hydroxyapitite pellet.

After correcting for non-specific binding, IC₅₀ values were determined. The IC₅₀ value is defined as the concentration of competing ligand needed to reduce specific binding by 50%. The IC₅₀ value was determined graphically from a loglogit plot of the data. The K_{d} values were determined by application of the Cheng-Prussof equation to the IC₅₀ values, the labeled ligand concentration and the K_{d} of the labeled ligand.

The results of ligand binding assay are expressed in K_{d} numbers. (See Chena et al. Biochemical Pharmacology Vol. 22 pp 3099-3108, expressly incorporated herein by reference.) The receptor affinity (K_{D} in nM) was greater than 10⁴ at all receptors for tazarotene. Tazarotenic acid, the parent compound of tazarotene, binds to RAR_{α}, RAR_{β}, and RAR_{γ} receptors with K_{D} values of 901±123 nM, 164±48 nM, and 353±37 nM, respectively. Binding data for tazarotenic acid is expressed as the mean and standard deviation. Since tazarotenic acid is more than about ten times as active as tazarotene (ie the binding constant is more than about ten times lower), this data demonstrates that tazarotene is a prodrug of the active drug tazarotenic acid.

### Example 1

### Microsphere Preparation

Poly(lactide-co-glycolide) 75:25 microspheres were prepared with a tazarotene loading of 10% w/w according the amounts in the table below.

Formula: Five-Gram Batch Size

| Component | Use | Quantity | |
|---|---|---|---|
| Phase I | | | |
| Polyvinyl Alcohol (PVA) | Stabilizer | 47.5 | grams |
| Purified Water | Solvent | 1600 | mL |
| Phase II | | | |
| Tazarotene | Active | 0.5 | (10%) |
| Poly lactide-co-glycolide | Polymer/Vehicle | 4.50 | grams |
| Methylene Chloride | Solvent | 300 | mL |

Phase I
In a five-liter beaker a solution of 3.0% PVA was prepared using a high shear impeller and a stirring rate of 400 to 500 rpm at 80° C. Once the PVA was in solution, the stirring rate was reduced to 200 RPM to minimize foaming.
Phase II
Poly(lactide-co-glycolide (PLGA) was then dissolved in the methylene chloride at room temperature. Once the PLGA was in solution, tazarotene was added and brought into solution also at room temperature.

Microspheres were then prepared using a solvent evaporation technique. Phase I solution was vigorously stirred at room temperature while slowly adding Phase II solution. The emulsion was then allowed to stir over 48 hours to remove the methylene chloride. The microspheres were then rinsed and finally freeze dried. The microspheres were frozen at −50° C., then freeze dried for at least 12 hours at a 4 mbar minimum pressure (400 Pa).

The freeze-dried microspheres were then sterilized by gamma irradiation at a dose of 2.5 to 4.0 mRad at 0° C. Temperature was maintained in the 0° C. cartons by the use of cold packs.

### Example 2

An aqueous suspension of tazarotene was prepared by adding tazarotene to isotonic phosphate buffered saline, pH 7.4 (IPBS) at room temperature. Twenty microliters of polysorbate 80® was added to the mixture. Finally, the tazarotene was dispersed by agitation to produce a uniform suspension of 20 mg/mL tazarotene in IPBS at room temperature.

### Example 3

An olive oil solution of tazarotene was prepared by simple addition of tazarotene to olive oil at room temperature. The mixture was vortexed at room temperature until the tazarotene was in solution. The final concentration of tazarotene was 20 mg/mL.

### Example 4

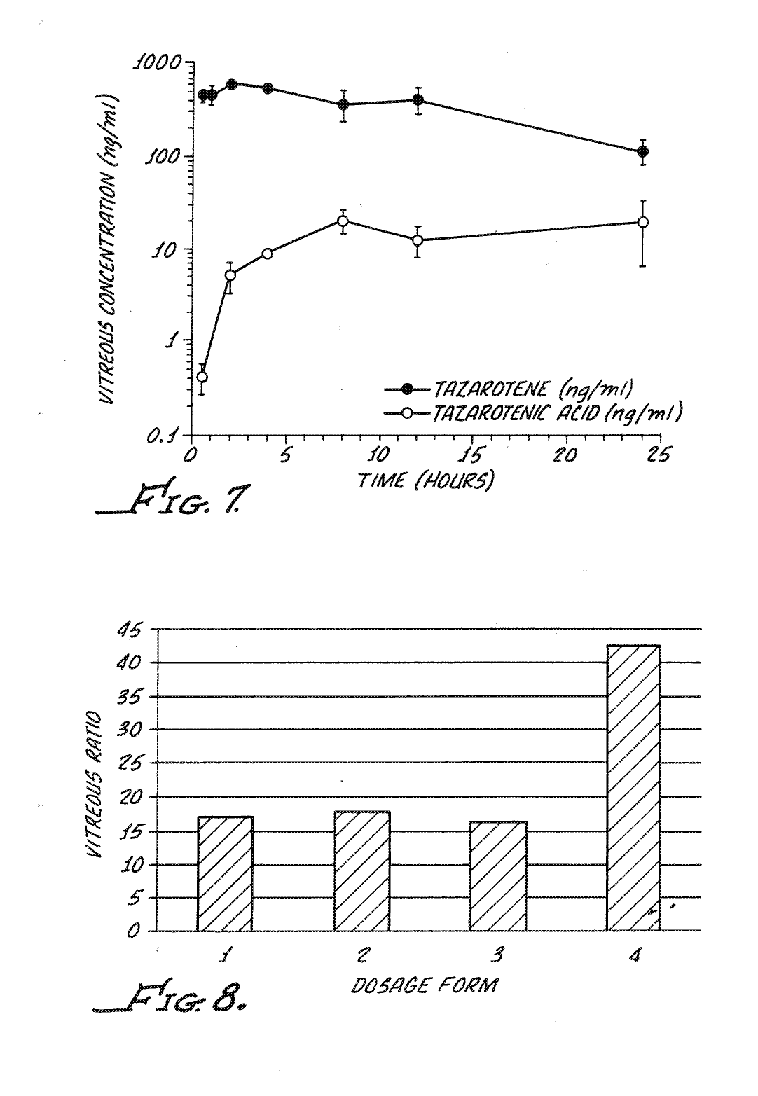

General disposition of tazarotene and tazarotenic acid resulting from intraocular and subconjunctival administration of tazarotene was assessed. Albino rabbits were dosed via intraocular injection with 1.25 μg of tazarotene. Injection was made mid-vitreous. After dosing the vitreous, retina and aqueous humor concentrations of tazarotene and tazarotenic acid were determined at 0.5, 1, 2, 4, 8, 12 and 24 hours post dosing. Turning to FIG. 7, the data clearly demonstrates that tazarotenic acid is generated from tazarotene in the vitreous where the concentration asymptotically approaches approximately 10 ng/ml. The data shows that the maximal vitreous concentration of tazarotenic acid obtainable after direct intraocular implantation is 10 ng/ml. Tazarotenic acid is eliminated in an apparent first order process from the vitreous with a half-life of 4.24 hours after midvitreous dosing of 1.25 μg of tazarotenic acid.

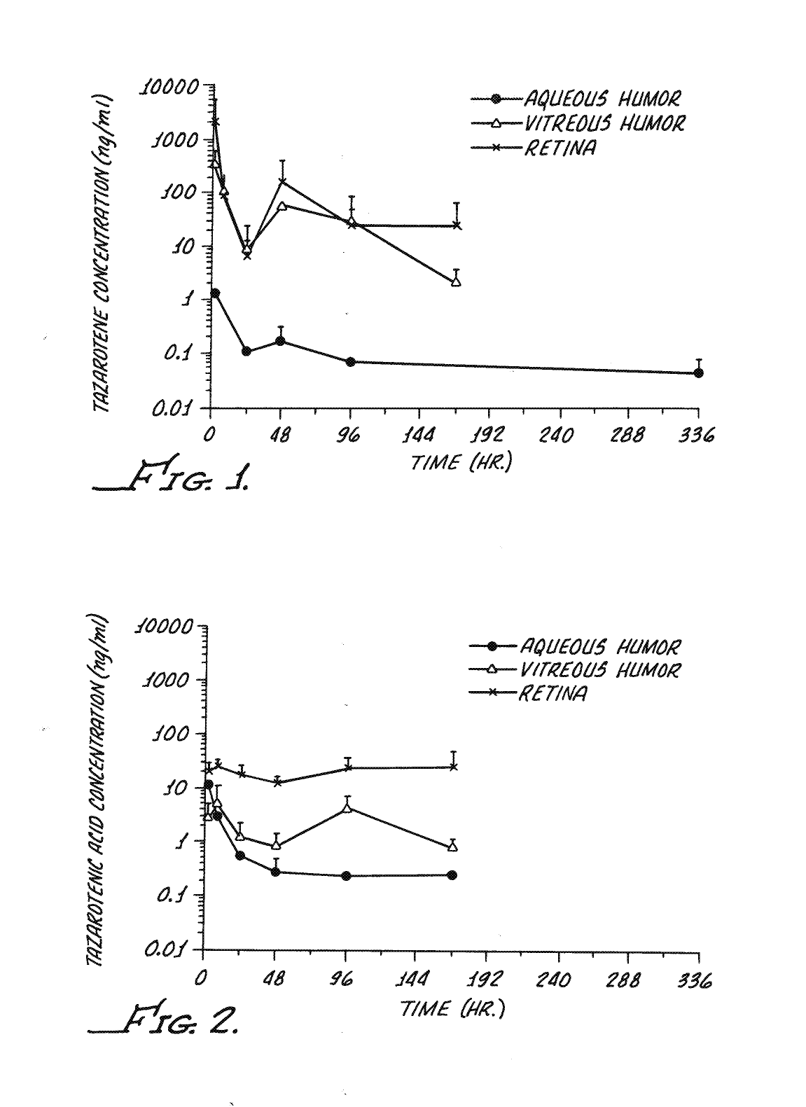

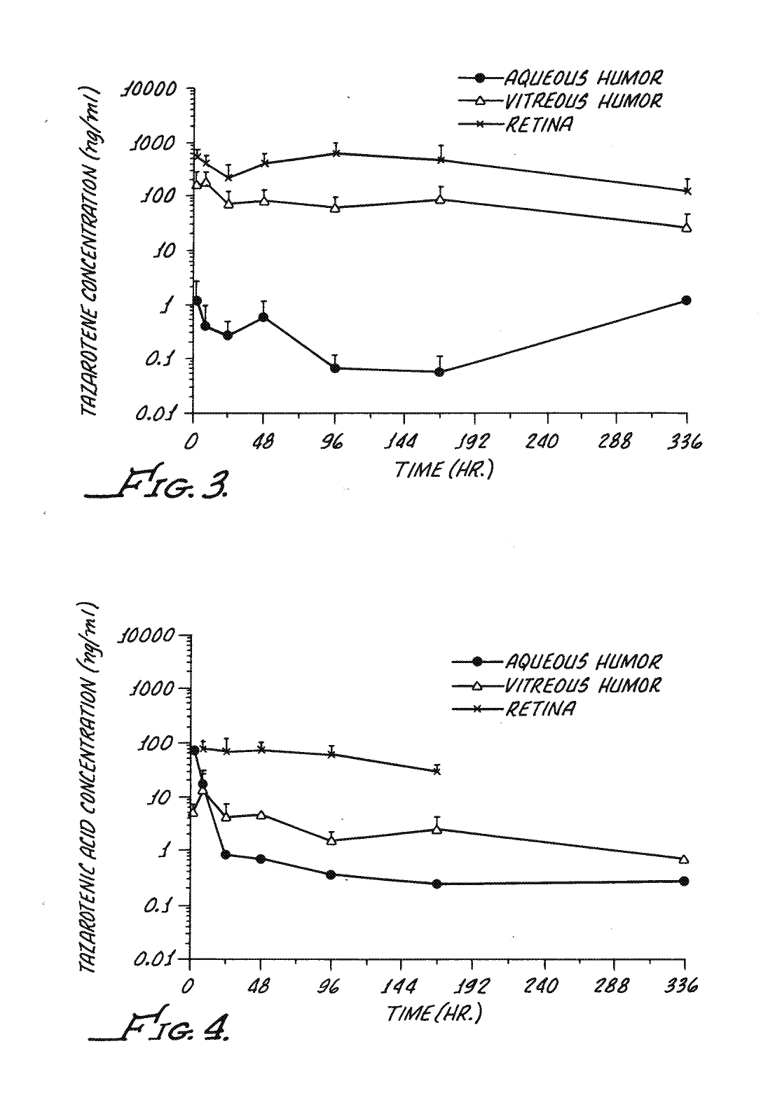

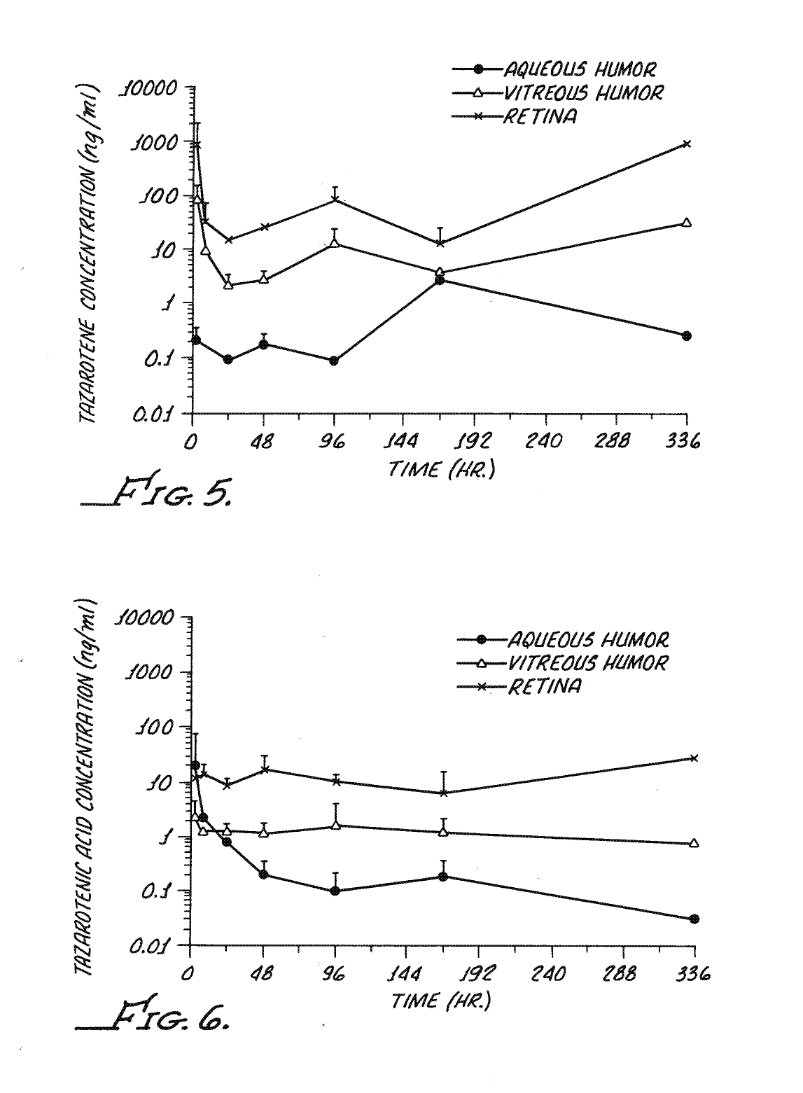

Tazarotene was also dosed in the subconjunctival space. Three dosage forms were evaluated: the tazarotene aqueous suspension described in Example 2 (50 μl of the solution, 1 mg tazarotene), tazarotene olive oil solution described in Example 3 (50 μl mg of the solution, 1 mg of tazarotene), and the tazarotene poly(lactide-co-glycolide) microsphere suspension described in Example 1. After dosing, the vitreous, retina and aqueous humor concentrations of tazarotene and tazarotenic acid were determined at 2, 8, 24, 48, 96, 168 and 336 hours post dosing (see FIGS. 1-8). These measurements showed that subconjunctival administration achieved significant levels of tazarotene and tazarotenic acid in the ocular tissues. More importantly, the ratio of tazarotene to tazarotenic acid was significantly lower than that obtained by injection of tazarotene directly into the vitreous, as shown in FIG. 8, indicating higher conversion of the prodrug to the active drug by this method of administration. The vitreous concentration data is summarized in Table 1. In Table 1 the mean vitreous concentration refers to average vitreous concentration observed from zero to one hundred sixty-eight hours post dosing. The mean vitreous concentration at each time point was used to calculate the overall vitreous mean concentration over the 168 hours for a given route of administration and dosage form. The vitreous concentration time profiles are summarized in FIGS. 1-7. In summary, the data clearly shows a more efficient delivery of tazarotenic acid from subconjunctival delivery compared with intravitreal delivery. It is also important to note that concentrations of the retinoids tazarotene and tazarotenic acid were maintained at low effective levels for a period of 336 hours (2 weeks).

TABLE 1
Vitreous Concentrations of Tazarotene and
Tazarotenic Acid after Intravitreal and
Subconjunctival Dosing.

| | Mean | Mean Vitreous | Tazarotene/ |
|---|---|---|---|
| | Vitreous | Concentration | Tazarotenic |
| | Concentration | Tazarotenic | Acid |
| Dosage Form | Tazarotene | Acid | Ratio |
| Intravitreal Injection | 417.0 | 9.9 | 42.0 |
| (1.25 μg) | | | |
| Subconjunctival | 42.0 | 2.5 | 16.8 |
| Suspension (1 mg) | | | |
| Subconjunctival | 21.9 | 1.4 | 16.1 |
| Microspheres (1 mg) | | | |
| Subconjunctival Oil | 96.2 | 5.43 | 17.7 |
| Solution (1 mg) | | | |

### Example 5

A dose of tazarotene (1 mg) contained in the poly(lactide-co-glycolide) microsphere suspension of Example containing 1 is injected subconjunctivally into a patient suffering from retinitis pigmentosa. Maintenance of vision or a slowing of the progression of vision loss is observed for the duration of treatment.

### Example 6

A dose of tazarotene (1 mg) contained in the poly(lactide-co-glycolide) microsphere suspension of Example containing 1 is injected subconjunctivally into a patient suffering from proliferative vitreal retinopathy. Traction retinal detachment is prevented or the rate of traction retinal detachment is reduced through treatment.

### Example 7

A dose of tazarotene (1 mg) contained in the poly(lactide-co-glycolide) microsphere suspension of Example containing 1 is injected subconjunctivally into a patient suffering from age related macular degeneration. Maintenance of vision or a slowing of the progression of vision loss is observed for the duration of treatment. Resolution of symptoms or a slowing in the progression of symptoms is achieved during therapy.

### Example 8

A dose of all-trans retinyl palmitate (1 mg) contained in the poly(lactide-co-glycolide) microsphere suspension of Example containing 1 is injected subconjunctivally into a patient suffering from retinitis pigmentosa. Maintenance of vision or a slowing of the progression of vision loss is observed for the duration of treatment.

What is claimed is:

1. A method of sustained delivery of an active drug to a posterior part of an eye of a mammal to treat a disease or condition of the eye affecting said mammal, wherein said disease or condition of the eye can be treated by the action of said active drug upon said posterior part of the eye, comprising administering an effective amount of an ester prodrug of the active drug subconjunctivally or periocularly to the eye, wherein the active drug is a prostaglandin, alpha-2-adrenergic agonist, beta adrenoreceptor antagonist, dopaminergic agonist, cholinergic agonist, tyrosine kinase inhibitor, corticosteroid, NMDA antagonist, anti-cancer drug, or antihistamine.

2. The method of claim 1 wherein the prodrug is contained in a polymeric microparticle system designed to enhance the sustained-delivery of said active drug.

3. The method of claim 2 wherein said polymeric microparticle system is a poly(lactide-co-glycolide) microsphere suspension.

4. The method of claim 1 wherein said posterior part of the eye comprises the uveal tract, vitreous, retina, choroid, optic nerve, or retinal pigmented epithelium.

5. The method of claim 1 wherein said disease or condition of the eye is retinitis pigmentosa, proliferative vitreal retinopathy, age-related macular degeneration, diabetic retinopathy, diabetic macular edema, retinal detachment, retinal tear, or uveitis.

6. The method of claim 1 wherein the prodrug is administered via injection.

7. The method of claim 1 wherein administration of the prodrug is subconjunctival.
